(19) United States
(12) Patent Application Publication
(10) Pub. No.: US 2026/0014171 A1
(43) Pub. Date: Jan. 15, 2026

(54) COMPOSITION FOR THE USE IN THE TREATMENT OF POLYCYSTIC OVARY SYNDROME

(71) Applicant: LO.LI. PHARMA S.R.L., Rome (IT)

(72) Inventors: Vittorio UNFER, Rome (IT); Virginia UNFER, Rome (IT); Michele RUSSO, Rome (IT)

(73) Assignee: LO.LI. PHARMA S.R.L., Rome (IT)

(21) Appl. No.: 19/139,650

(22) PCT Filed: Feb. 15, 2024
(86) PCT No.: PCT/EP2024/053814

§ 371 Date: Jun. 16, 2025

(30) Foreign Application Priority Data
Feb. 17, 2023 (IT) 102023000002769
Mar. 14, 2023 (IT) 102023000004827

(51) Int. Cl. A61K 31/58; A61K 9/20; A61K 31/593; A61K 36/8945; A61K 38/38

(52) U.S. Cl. CPC A61K 31/58; A61K 9/2009; A61K 9/2013; A61K 9/2054; A61K 9/2059; A61K 31/593; A61K 36/8945; A61K 38/38

(57) ABSTRACT

The composition and/or combinations includes diosgenin and at least one other agent selected from vitamin D and α-lactalbumin and its use in the regularization of the menstrual cycle and in the reduction of ovarian cysts, in particular in women affected with polycystic ovarian syndrome (PCOS) phenotype D.

### FIELD OF INVENTION

The present invention regards a composition and/or combination comprising diosgenin and at least one of the following agents: vitamin D and α-lactalbumin, for use in the regularization of the menstrual cycle and the reduction of ovarian cysts, particularly in women affected by polycystic ovary syndrome (PCOS) phenotype D.

### STATE OF THE ART

PCOS is common pathology affecting 5 to 15% of women of reproductive age and is characterized by endocrinological and often metabolic alterations in the affected patients [1].

The diagnosis of PCOS is governed by the Rotterdam criteria which define four distinct patient phenotypes.

Phenotype A (Classical or canonical PCOS), are patients characterized by clinical or biochemical evidence of hyperandrogenism, evidence of oligo-ovulation, and ultrasound evidence of cystic ovaries; Phenotype B (hyperandrogenic anovulatory PCOS), are patients characterized by clinical or biochemical evidence of hyperandrogenism and evidence of oligo-ovulation; Phenotype C (ovulatory PCOS), are women characterized by clinical or biochemical evidence of hyperandrogenism and ultrasound evidence of cystic ovaries; Phenotype D (non-hyperandrogenic PCOS) are instead, patients characterized by evidence of oligo-ovulation and ultrasound evidence of cystic ovaries [2].

The etiopathology of PCOS is a much-debated subject as the diverse aspects of the pathology have not been completely clarified, despite much study over the years. This has resulted in a diversification of the therapeutic approaches used in the treatment of PCOS, that change according to the specialist who prescribes treatment. Regardless, following the diagnosis of PCOS, the therapy that is prescribed does not take into consideration the phenotype of the given patient [3]. The present invention instead, proposes the introduction of a therapy that is differentiated, specifically according to the characteristics of the patient, in particular offering a completely new therapy for women who present with phenotype D.

Women affected by phenotype D of PCOS do not demonstrate any signs of hyperandrogenism but rather present with ultrasound evidence of cystic ovaries and anovulation and consequent disruption to the menstrual cycle. According to an estimate published in 2020, considering a reference population, the prevalence of phenotype D amounted to 20% of the patient population [4]. These women are treated in the same manner as the other phenotypes of PCOS and are initially recommended to undertake a change in lifestyle to avoid eventual pharmacological treatments. Regular physical exercise, a balanced low-calorie, fat and sugar diet with a balanced nutritional intake, which keeps body weight under control is recommended for women with this syndrome. When this approach is not sufficient, the typical recommended therapies are principally comprised of contraceptive devices such as OCPs (oral contraceptive pills) which are prescribed for the regularization of the menstrual cycle and for the reduction of aesthetic signs of hyperandrogenism such as acne, hirsutism, and hair thinning. While this treatment represents the most common approach for PCOS, OCPs do not have a rational application in phenotype D patients, as these patients do not present evidence of hyperandrogenism. Furthermore, numerous controversies are associated with the use of OCPs, above all due to the undesired effects deriving from long term treatment that results by weight gain leading to alterations in cardio-metabolic parameters [5]. An alternative therapeutic approach utilized in this pathology comprises of the treatments that seek to improve metabolic alterations frequently observed in women with PCOS, including obesity, insulin-resistance, and dyslipidemia. The administration of pharmaceuticals indicated for weight loss (ligralutide, lorcaserin) or therapies which use insulin-sensitizing molecules (metformin and inositol) are known to reduce circulating androgen levels in the blood, improve the metabolic profile, and induce a regularization of the menstrual cycle [3, 6]. Although these molecules have had success for many years in the treatment of this pathology, it is important to consider that phenotype D patients do not present with hyperandrogenism and do not always demonstrate metabolic alterations. Therefore, the approaches described above do not represent the treatment of choice for a subset of PCOS patients who require a more personalized and functional therapy. A therapeutic alternative designed and developed for women with phenotype D PCOS is represented within the present invention, which aims to offer a real possibility of improving the pathological conditions in these women.

### SUMMARY OF THE INVENTION

The present invention describes a combination of three natural molecules, that alone, do not have therapeutic value in the treatment of phenotype D PCOS, i.e., diosgenin, or in the form of an extract of a plant belonging to the Dioscoreaceae family, preferably in the form of an extract Dioscorea villosa, vitamin D, and α-lactalbumin, that together have demonstrated a novel activity in this phenotype of PCOS.

The described object of the invention is a composition comprising diosgenin or a salt or derivative thereof and at least one of the following agents: vitamin D, and α-lactalbumin.

Preferably the diosgenin is comprised of powdered plant parts or an extract of said parts of the plants, in which said powdered plant parts or extracts are obtained preferably from plants belonging to the Dioscoreaceae, preferably Dioscorea villosa, Dioscorea alata, Dioscorea bulbifera, Dioscorea composita, Dioscorea oppostifolia, Dioscorea polystachy, Dioscorea hirticaulis, Dioscorea nipponica, Dioscorea zingibergensis, Dioscorea deltoidea, Dioscorea collettii, Dioscorea semptemloba, more preferably said powdered plant parts or extracts comprise diosgenin in an amount of between approximately 1% to 50% by weight, preferably between approximately 1% to 30% by weight, preferably approximately 20% by weight, about 15% by weight, about 10% by weight or about 6% by weight.

Preferably said composition comprises:
a) Plant parts, preferably powdered plant parts or extracts obtained from Dioscorea villosa in an amount between 1 and 600 mg, preferably between 200 mg and 600 mg, preferably between 200 mg and 400 mg, preferably between 250 mg and 350 mg, more preferably in the amount of 300 mg;
b) Vitamin D in an amount between 1 and 50 μg (40 to 2000 UI), preferably in an amount between 10 to 40 μg (400 to 1600 UI), preferably in an amount between 20 and 30 μg (800 to 1200 UI), more preferably in an amount of 25 μg (1000 UI); and/or
c) α-lactalbumin in an amount between 1 and 100 mg, preferably in an amount between 25 and 75 mg, preferably in an amount between 40 and 60 mg, preferably in the amount of 50 mg.

Still preferably the composition comprises:
a) Parts of plants, preferably powdered parts of plants or an extract thereof obtained from Dioscorea villosa in an amount comprised from 1 to 600 mg, preferably from 200 mg to 600 mg, preferably from 200 mg to 400 mg, preferably from 250 mg and 350 mg, more preferably in the amount of 300 mg;
b) Vitamin D in an amount comprised between 1 and 50 μg, i.e. from 40 to 2000 UI, preferably in an amount comprised between 10 and 40 μg, i.e. from 400 to 1600 UI, preferably in the amount comprised between 20 and 30 μg, i.e. from 800 and 1200 UI, more preferably in the amount of 25 μg i.e. 1000 UI; and
c) α-lactalbumin in an amount comprised between 1 and 100 mg, preferably in an amount between 25 and 75 mg, preferably comprising α-lactalbumin in the amount between 40 and 60 mg, preferably comprising α-lactalbumin in the amount of 50 mg.

Preferably said composition comprises diosgenin in an amount from 0.2 mg to 120 mg, preferably from 40 mg to 120 mg, preferably from 40 mg to 80 mg, preferably from 50 mg to 70 mg, preferably comprising about 60 mg.

Preferably said composition comprises diosgenin in an amount from 0.06 mg and 36 mg, preferably from 12 mg to 24 mg, preferably from 15 mg to 21 mg, preferably in the amount of about 18 mg.

In a preferred form the invention is a composition as described above, whereby vitamin D is selected from: ergocalciferol, cholecalciferol, calciferol, calcitriol, calcifediol and/or alpha-lactalbumin is selected from α-lactalbumin, lactalbumin hydrolysates, whey protein isolate.

A further object of the invention is a composition comprising diosgenin or a salt or derivative thereof and at least of the following agents: vitamin D and α-lactalbumin, in addition to a excipient or diluent, preferably for oral and/or topical administration, preferably in the form of a tablet, a hard capsule, a soft gel capsule, a powder, a syrup, a tablet, a lozenge, a bar, a gel, a cream, an ointment, preferably said excipient or diluent is one of the following: calcium phosphate, dicalcium phosphate, microcrystalline cellulose, silicon dioxide, sucrose, gum arabic, corn starch, medium chain triglycerides, tricalcium phosphate, cross-linked sodium carboxymethylcellulose, hydroxypropyl methylcellulose, polyethylene glycol, titanium dioxide, polyvinylpyrrolidone, talc, erythritol, xylitol, steviol glycosides and sucralose, magnesium salt of fatty acids, preferably said magnesium salt of fatty acids is magnesium stearate.

A further object of the invention is a composition comprising diosgenin or a salt or derivative thereof and at least of the following agents: vitamin D and α-lactalbumin as defined above for use in the regularization of the menstrual cycle and reduction of ovarian cysts in women affected by polycystic ovary syndrome (PCOS), preferably PCOS phenotype D.

A further object of the invention is a dermo-cosmetic product or medical device or supplement or dietary product, or a formulation for topical use containing the composition of the invention.

A further object of the invention is the non-therapeutic use of a composition comprising diosgenin or a salt or derivative thereof and at least of the following agents: vitamin D and α-lactalbumin or of a dermo-cosmetic product or medical device or supplement or dietary product or a formulation for topical use containing said composition, in the nutraceutical sector or as a base ingredient in the preparation of supplements and/or devices for topical use and/or dietary products and/or food for specific medical purposes and/or agents for the regularization of the menstrual cycle and the reduction of ovarian cysts, preferably for the regularization of the menstrual cycle and the reduction of ovarian cysts in women affected by polycystic ovarian syndrome (PCOS), preferably PCOS phenotype D.

A further object of the invention is a combination comprising diosgenin or a salt or derivative thereof and at least of the following agents: vitamin D and α-lactalbumin for use in regularization of the menstrual cycle and the reduction of ovarian cysts, preferably for the regularization of the menstrual cycle and the reduction of ovarian cysts in women affected by polycystic ovarian syndrome (PCOS), preferably PCOS phenotype D.

Preferably in said combination the diosgenin is comprised of powdered plant parts or an extract of said parts of the plants, in which said powdered plant parts or extracts are obtained preferably from plants belonging to the Dioscoreaceae, preferably Dioscorea villosa, Dioscorea alata, Dioscorea bulbifera, Dioscorea composita, Dioscorea oppostifolia, Dioscorea polystachy, Dioscorea hirticaulis, Dioscorea nipponica, Dioscorea zingibergensis, Dioscorea deltoidea, Dioscorea collettii, Dioscorea semptemloba, more preferably said powdered plant parts or extracts comprise diosgenin in an amount of between approximately 1% to 50% by weight, preferably between approximately 1% to 30% by weight, preferably between approximately 1% to 20% by weight, about 20% by weight, about 15% by weight. Still preferably said powdered plant parts or extracts comprise diosgenin in an amount of between approximately 1% to 15% by weight, preferably between approximately 1% to 10% by weight, preferably between approximately 5% to 10% by weight, more preferably approximately 6% by weight.

Preferably said combination comprises:
a) Plant parts, preferably powdered plant parts or extracts obtained from Dioscorea villosa in an amount between 1 and 1200 mg/day, preferably between 200 mg/day and 1000 mg/day, preferably between 200 mg and 800 mg/day, preferably between 400 mg/day and 800 mg/day or between 300 and 700 mg/day, more preferably in the amount of 600 mg/day, still preferably in the amount of 300 mg/day;
b) Vitamin D in an amount between 1 and 100 μg/day (40 to 4000 UI/day), preferably in an amount between 20 to 80 μg/day (800 to 3200 UI/day), preferably in an amount between 25 and 60 μg/day (1000 to 2400 UI/day), more preferably in an amount of 50 μg/day (2000 UI/day); and/or
c) α-lactalbumin in an amount between 1 and 200 mg/day, preferably in an amount between 25 and 175 mg/day, preferably in an amount between 50 and 150 mg/day, preferably in an amount between 75 and 125 mg/day, preferably in the amount of 100 mg/day.

In a preferred form of the invention, said combination comprises diosgenin in an amount between 0.2 mg and 240 mg/day, preferably between 40 mg and 200 mg/day, preferably between 80 mg and 200 mg/day, preferably between 80 mg and 160 mg/day, preferably about 120 mg/day.

In a preferred form of the invention, said combination comprises: diosgenin in an amount between 0.06 mg/day to 72 mg/day, preferably from 12 mg/day to 48 mg/day, preferably from 15 mg/day to 42 mg/day, preferably comprising approximately 18 mg/day, more preferably about 36 mg/day.

Preferably in said combination, vitamin D is selected from: ergocalciferol, cholecalciferol, calciferol, calcitriol, calcifediol and/or alpha-lactalbumin is selected from α-lactalbumin, lactalbumin hydrolysates, whey protein isolate.

Preferably in said combination, diosgenin and the other agent or agents are administered simultaneously and/or separately and/or orally and/or applied topically once and/or twice daily.

The invention will now be illustrated with the aid of non-limiting examples with reference to the following figures.

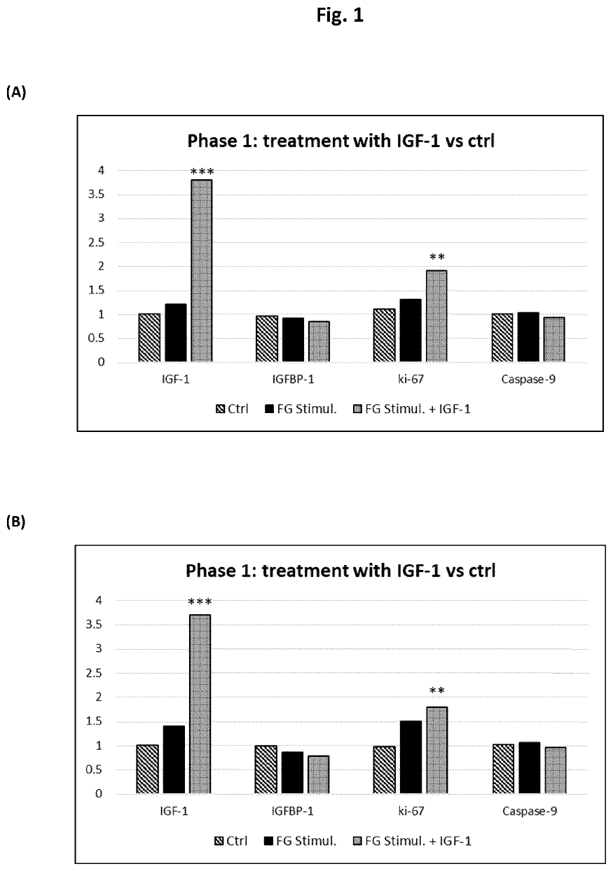

FIG. 1. Phase 1 treatment with IGF1 vs control. (A) mRNA results: The figure displays data on the gene expression of IGF-1, IGFBP-1, ki-67 and caspase-9 following stimulation of bovine cell cultures with FSH followed by LH+insulin, and FSH followed by LH+insulin and subsequently the addition of exogenous IGF-1, compared with the untreated control. Exogenous IGF-1 stimulus induces a significant increase in IGF-1 and ki-67 expression, while other changes were not significant. (B) Protein results. The figure displays data on the quantification of IGF-1, IGFBP-1, ki-67 and caspase-9 proteins, following stimulation of bovine cell cultures with FSH followed by LH+insulin, and FSH followed by LH+insulin and subsequently the addition of exogenous IGF-1 compared with the untreated control. Stimulation with IGF-1 induces a significant increase in IGF-1 and ki-67, while the other changes were not significant. Legend: *=p<0.05, **=p<0.01, ***=p<0.001

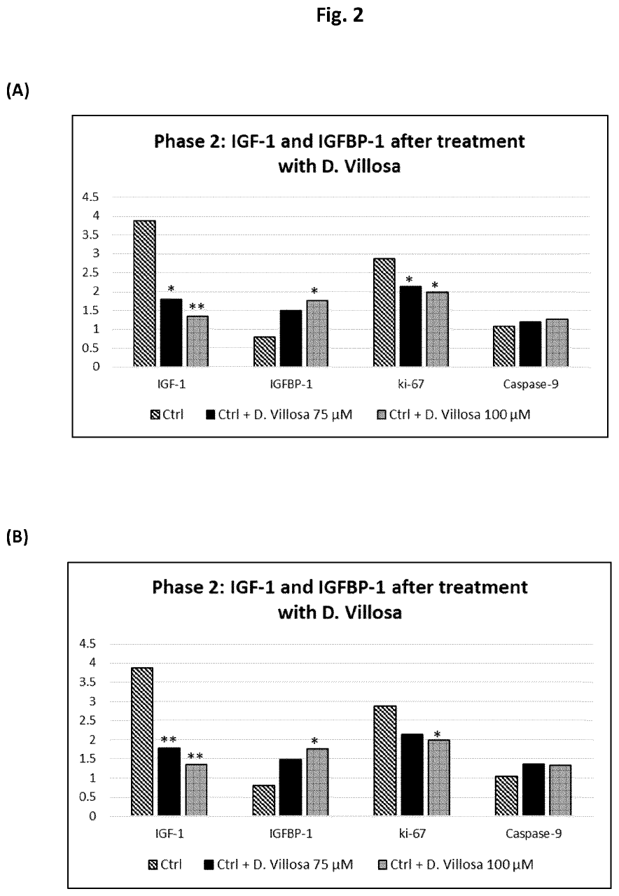

FIG. 2. Phase 2 IGF-1 and IGF-BP-1 after treatment with Dioscorea villosa. (A) mRNA results: The figure displays the gene expression data of IGF-1, IGFBP-1, ki-67 and caspase-9, following stimulation of bovine cell cultures with a control (stimulation with FSH followed by LH+insulin and subsequently the addition of exogenous IGF-1), control+Dioscorea villosa 75 UM for 24 h, and control+Dioscorea villosa 100 μM for 24 h. The addition of Dioscorea villosa alone significantly reduces the expression of IGF-1 and ki-67 proportionally to the dose administered, and significantly increases the expression of IGFBP-1 in an equally proportional manner. (B) Protein results: The figure displays data on the quantification of IGF-1, IGFBP-1, ki-67 and caspase-9 proteins, following stimulation of bovine cell cultures with a control (stimulation with FSH followed by LH+insulin and subsequently the addition of exogenous IGF-1), control+Dioscorea villosa 75 μM for 24 h, and control+Dioscorea villosa 100 μM for 24 h. The addition of Dioscorea villosa alone significantly reduces IGF-1 expression in proportion to the dose administered, significantly increases the IGFBP-1 protein only in the case of the 100 μM Dioscorea group, and significantly reduces ki-67 also in the case of the Dioscorea 100 μM group. Legend: *=p<0.05, **=p<0.01, ***=p<0.001

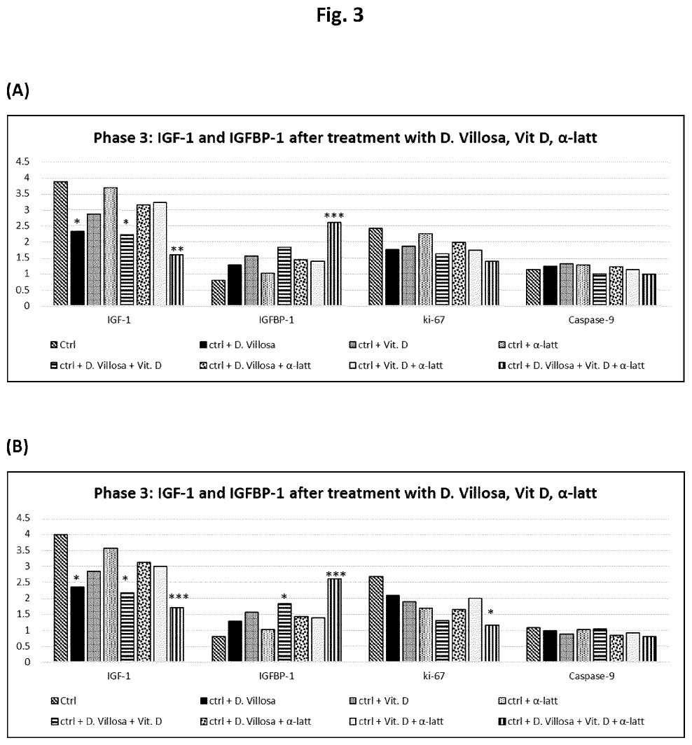

FIG. 3. Phase 3 IGF-1 IGF-1 and IGF-BP-1 after treatment with Dioscorea villosa, Vitamin D and alpha-lactalbumin. (A) mRNA results: The figure displays data on the gene expression of IGF-1, IGFBP-1, ki-67 and caspase-9, following stimulation of bovine cell cultures with a control (stimulation with FSH followed by LH+insulin and subsequently the addition of exogenous IGF-1), and administration of Dioscorea villosa, Vitamin D and α-lactalbumin as single raw materials or in combination. The administration of Dioscorea villosa alone and in combination with Vitamin D significantly reduces the expression of IGF-1, but the combination of the 3 raw materials is more effective in reducing IGF-1. Similarly, the combination of the 3 raw materials induces a significant increase in IGFBP-1. (B) Protein results: The figure displays data on the quantification of IGF-1, IGFBP-1, ki-67 and caspase-9 proteins, following stimulation of bovine cell cultures with a control (stimulation with FSH followed by LH+insulin and subsequently the addition of exogenous IGF-1), and administration of Dioscorea villosa, Vitamin D and α-lactalbumin as single raw materials or in combination. The administration of Dioscorea villosa alone and in combination with Vitamin D significantly reduces the IGF-1 protein, but the combination of the 3 raw materials is more effective in reducing IGF-1. The administration of Dioscorea villosa alone and in combination with Vitamin D significantly increases the IGFBP-1 protein, but the combination of the 3 raw materials is more effective in increasing IGFBP-1. In addition, the combination of the 3 raw materials significantly reduces ki-67. Legend: *=p<0.05, **=p<0.01, ***=p<0.001

### DETAILED DESCRIPTION OF THE INVENTION

PCOS is often characterized by endocrine and metabolic disorders in the majority of patients, that manifest with characteristic phenotypic features such as acne, hirsutism, and hyperandrogenism. However, since phenotype D of PCOS does not present with hyperandrogenism, debate over its origin, resulting in much controversy. Furthermore, questions have been raised regarding whether generic strategies may deliver improvements for this category of patients. Studies have demonstrated that this phenotype appears to be associated in some cases with alterations in the glucose and the insulinemic profile, but this seems to have little correlation with problems of anovulation and the formation of ovarian cysts. This makes the need of an ad hoc treatment for the aforementioned subclass of patients, with a specific mode of action tailored towards disorders that these women present even more necessary.

In the study of this particular phenotype of PCOS during this present invention, is evidenced an innovative therapeutic function derived from the association of three natural molecules that alone do not have curative value in the field of this syndrome. The molecules that are described within the present invention are diosgenin, preferably in the form of a plant extract from the Dioscoreaceae family, preferably in the form of an extract from Dioscorea villosa, vitamin D, and α-lactalbumin, that together have demonstrated a novel activity in this phenotype of PCOS.

The present invention, in fact, was developed in a serendipitous manner as the inventors discovered that the cause of this phenotype may be a condition of relative hyper-estrogenism, which prevents correct ovulation and causes the formation of ovarian cysts.

Estrogens regulate diverse processes with the female body, and they are important modulators of reproductive physiology. However, excessive activation of estrogen receptors and an elevated synthesis of estrogen-dependent enzymes is associated with the onset of pathological conditions such as PCOS, endometriosis, and ovarian and breast cancer [7].

Estrogen receptors (ER) exist as two types: ER-a which are those primarily expressed within the uterus, ovary and breast and ER-B, that although expressed to a lesser degree than ER-a in the ovarian region, they seem heavily involved in the proliferative effect that is characteristic of endometriosis.

The main types of endometrial cells on which estrogens act are epithelial and stromal cells. The mitotic active and strong proliferative effect that estrogens induce in these types of cells are two main characteristics of the follicular or proliferative phase of the menstrual cycle. Elevated estrogen levels promote an increased expression of the two receptors ER-a and ER-B, reaching a peak late in the follicular phase. At the same time estradiol increases the levels of progesterone receptors, which in turn decreases the expression of ER-a and ER-B receptors, inducing the onset of the luteal phase also known as the secretory phase. It is through this mechanism that the begins the process of cellular differentiation and reprograming within the endometrium.

The altered profile of the expression of estrogen receptors is one of the markers overrepresented in the endometrium of women with PCOS phenotype D, and this endometrial characteristics in these women may explain the occurrence of common pathological manifestations such as chronic anovulation.

This action is significant, as an increase in estrogens and it their receptors has allowed the authors of the present invention to focus their attention on the molecular aspect that suggests a hyperestrogenic condition in the women with PCOS phenotype D.

The starting point was an accurate study of the mechanisms that are involved in ovulation and consequently in the female menstrual cycle and the various molecules that intervene in this delicate process. Furthermore, attention was paid to aspects that can alter the physiology of this process by examining all the experimental evidence found in both humans, and mouse models.

In this regard, recent evidence has demonstrated that in three dimensional follicles observed in mouse models, an excessive production of insulin growth factor 1 (IGF-1) inhibits correct follicle growth. The activity of IGF-1 seems to favor cellular proliferation without inducing apoptosis unless administrated at very high doses. What to date has yet to be demonstrated, is that this factor is strongly expressed in women with PCOS phenotype D and that when the expression of reaches high levels, this causes further production of estrogen which inhibits the physiological ovulation process.

From a molecular point of view, IGF-1 regulates diverse intra-ovarian processes and is a fundamental pancreatic and endocrine factor expressed during folliculargenesis. It carries out its biological function by binding to the transport protein IGF binding protein 1 (IGFBP-1) or to its receptor IGF-1R. The interests of the inventors focus primarily on the protein IGFBP-1 that, other than acting as a transport protein, modulates the action of IGF-1 at the cellular level and is also able to bind to IGF-1 receptors. The aspect that has been analyzed in detail is that IGFBP-1 is a progesterone-dependent protein. Therefore, in the follicular phase these women produce estrogens that increase as the follicle becomes pre-antral to reach a peak in the antral phase. This leads to a large increase in IGF-1 and this overexpression induces a strong proliferative effect at the ovarian and endometrial level in addition to further production of estrogens. In the luteal phase, however, the increase of progesterone triggers an increase in IGFBP-1 (secreted by the endometrium) that modulates the action of IGF-1 and accelerates cellular differentiation of the endometrium. Since the luteal phase is regulated by progesterone levels, high levels of progesterone are necessary to maintain the decidualization of the endometrium before menstruation.

Women affected by PCOS phenotype D, as previously mentioned, demonstrate alterations in the menstrual cycle, which almost always derives from a prolongation of the follicular phase, reflected in a lengthening of the time between period cycles. If it is assumed that the duration of the two phases is equivalent to 14 days and lengthening of the follicular phase induces a cycle that is less frequent in these women (>30-35 days).

The authors have discovered that insufficient levels of progesterone or the altered functionality of the corpus luteus seen in these patients with PCOS phenotype D reduces strongly the expression of IGFBP-1 which not being able to proficiently bind to IGF-1, gives rise to a strong proliferative effect due to excessive levels of IGF-1.

The explicit action of IGF-1 at the ovarian level is at its physiological peak during the pre-antral phase. However, in the pathological condition PCOS phenotype D, this strong proliferative effect is associated with a reduction in the activity of IGF-BP-1 continues, prolonging the follicular phase. This consequently induces the growth of follicles which do not reach maturity and subsequently form the characteristic ovarian cysts, in addition to an excessive thickening of the endometrium.

These molecular alterations induce a consequential anovulatory infertility that is one of these traits often observed in women with PCOS phenotype D, characterized by anovulatory cycles, often associated with anomalies in the endometrium. These aforementioned alterations can cause difficulties for women trying to conceive.

Women with PCOS phenotype D are characterized by chronic anovulation, as the lack of a regulatory effect of progesterone results in the endometrium being unable to enter the secretory or luteal phase and remains continuously exposed to the mitogenic stimulus of estrogen.

Women with PCOS display a deregulation of biomarkers associated with the secretory phase and endometrial receptivity [9]. In detail, the increase in expression of IGF-1 and the reduced expression of IGFBP-1, recognized as biomarkers of the decidualization process, induces an excessive growth of the endometrium and a consequent alteration in the menstrual cycle that often manifests as chronic anovulation.

For this reason, the idea of using a molecule such as diosgenin was proposed. Diosegnin, or in the the form of a plant extract comprising diosgenin, preferably said plant is a plant of the Dioscoreaceae family, more preferably said plant is Dioscorea villosa, that is typically used to cure disorders associated with menopause in women who are going through this particular stage of life. An important aspect that is not considered by the published data is that its application can not only treat menstrual cycle disorders but also contribute to the disappearance of the characteristic ovarian cysts observed in these women, in addition to providing excellent support for those wishing to conceive.

Dioscorea villosa also known as wild yam, it is a woodland medicinal herb, originating from the forest of north America. Its extract from the rhizome and the roots are famously used to counter the effects of menopause and it is indicated for the treatment of rheumatoid arthritis.

Furthermore, Dioscorea villosa is often recommended due to its known benefits in reducing chronic inflammation and dysmetabolism in affected women. Considering that these alterations are observed also in patients with PCOS phenotype D and that they could contribute to the etiopathogenesis of this syndrome, the authors discovered that the use of Dioscorea villosa is fundamental in the treatment of these patients.

An important characteristic for the purposes of this invention is that these women are often characterized by hypo-progesteronemia. With the administration of said diosgenin, or an extract that includes diosgenin, preferably with the administration of an extract of Dioscorea villosa that exploits the properties of dioseginin, in particular the progesterone simulating effect, which induces an increased expression of the IGFBP-1 protein and contributes to synchronize the signals of IGF-1 which in turn restores normal levels of estrogen and promotes correct ovulation.

Another new aspect that has not been attributed to this natural extract is that it could strongly contribute to the reduction of ovarian cysts that are characteristic of these women with PCOS phenotype D. In this sense the presented findings add another aspect of novelty, which consists of the combination of diosgenin, or in the form of an extract from Dioscorea villosa, with vitamin D and a/lactalbumin that significantly amplifies the beneficial effects of the extract of Dioscorea villosa.

The female reproductive system is complex as various molecules interact for the constant maintenance of current function. Of these molecules vitamin D is vital, such that if vitamin D levels are deficient, above all in specific moments, it may impede the successful start of a pregnancy. The activity of vitamin D is very similar to progesterone, that is fundamental for the physiology of the female menstrual cycle, and is a fundamental molecule for the process of embryo implantation [10].

It is estimated that around approximately a billion people worldwide have a deficiency of Vitamin D and numerous studies have indicated that this deficiency is associated with conditions frequently observed in women with PCOS, such as insulin-resistance, diabetes, dyslipidemia, anovulation, in addition to infertility.

It is thought that between 67% and 87% of women affected by PCOS have a deficiency in vitamin D, in particular in those women with a greater body mass index.

Various evidence has demonstrated that vitamin D can alter the signaling of anti-mullerian hormone (AMH) and the sensitivity to follicular stimulating hormone (FSH), in addition to modulating the production and release of progesterone from granulosa cells. This suggests vitamin D has a key role in oocyte follicular development and luteinization. In fact, in granulosa cells undergoing luteinization, vitamin D reduces the expression of AMH and FSH receptors. In the selection of the dominant follicle during the late follicular phase, the aforementioned follicle becomes less dependent on FSH signals and increasingly dependent on the signal of luteinizing hormone (LH). This process ends with the definite maturation of the follicle and consequently with ovulation. The expression of FSH and LH receptors in granulosa cells appear to be elevated in small immature follicles and reduce gradually with the progression towards oocyte maturation. Therefore, vitamin D stimulates the action of enzymes involved in steroidogenesis, thus promoting luteinization and improving the ovarian microenvironment.

The importance of vitamin D administration is even more critical in women with PCOS phenotype D that demonstrate a high body mass index and present metabolic conditions such as insulin resistance or dyslipidemia. These women are at an ever-increasing risk of developing vitamin D hypovitaminosis, because the hydrophobic chemical structure of vitamin D makes it easy to be internalized and subsequently it accumulates in body fat tissue, strongly decreasing the bioavailability within the human body and consequently within the ovary.

α-lactalbumin (α-LA), on the other hand, is a globular protein present in milk serum and produced by epithelial cells of the mammary gland. It is known that women affected by PCOS often suffer from dysmetabolism and this condition is frequently associated with alterations in the microbiota which has an important role in the pathogenesis of this syndrome.

Alterations in the composition of the microbiota, known also as dysbiosis, negatively influence inflammatory processes, and correlate with diverse chronic pathologies such as type 2 diabetes, and notably PCOS.

In women with PCOS, gastrointestinal dysbiosis reflects alterations in the biodiversity of the microbiota. Often these alterations can induce insulin-resistance, alterations in the menstrual cycle, and the morphology of the ovary. Furthermore, conditions such as obesity, in addition to the high fat diet commonly found in women with phenotype D, promote the growth of pro-inflammatory bacteria. These alterations weaken the tight junctions of the intestinal mucosa, in this way inflammatory mediators can spread systemically and trigger the immune system response [12].

Another aspect focused on by the authors is the fact that a dysbiosis of the gastric track can have repercussions such as a consequent alteration of the vaginal microbiota. This dysbiosis in turn can influence the menstrual cycle and is associated with the onset of chronic anovulation in patients with PCOS phenotype D. An alteration of the intestinal and vaginal flora, can further influence the regulation of free estrogen and potential cause an imbalance in the levels of estrogens, leading to relative hyperestrogenism which is identified as the therapeutic target of the present invention.

In this context, the role of α-LA is particularly important due to its prebiotic activity, in addition to its widely studied ability to improve the absorption of molecules such as inositol.

Therefore, the innovative aspect, which has not been previously described, is that α-LA when associated with vitamin D and diosgenin, or in the form of an extract, preferably in the form of an abstract of Dioscorea villosa, is able to enhance the cycle regularization effect, regulating the estrogenic imbalance in women with PCOS phenotype D, and a major reduction in ovarian cysts in respect to the observed effect of singular administration of either vitamin D or diosgenin alone. This novel action of α-LA has not been described previously and cannot be simply deduced through analysis of the present literature that regards this molecule.

Another interesting aspect of the present invention is that when vitamin D is combined with α-LA, the latter is able perform a carrier action to improve the absorption of vitamin D. In fact, the vitamin D and α-LA complex appears to represent a functional way to stabilize vitamin D, thus promoting its intake from vitamin D containing foods.

The innovation described within the present rationale has unexpectedly demonstrated that this action of α-LA on vitamin D, in the presence of diosgenin or in the form on an extract, preferably in the form of an extract of Dioscorea villosa, significantly amplifies the reduction of ovarian cysts that are characteristic of these women. This combination of elements is much more effective than the observable effects of each singular component, it is reasonable to consider that the present findings can provide a efficacious natural remedy for women with PCOS phenotype D.

Due to the associated actions in the present finding, the authors have observed an important reduction in ovarian cysts that are characteristic of patients with PCOS phenotype D. The ovarian cysts are sacs of variable dimensions (in the order of centimeters) that frequently form within the ovary of these women. As a rule, cysts, are part of natural physiological and are dependent on the menstrual cycle, usually benign and do not cause symptoms or disorders in the women which they appear. The majority of cysts regress spontaneously after a period of several months, without the necessity of therapy or surgery. In some cases, however, like in PCOS, cysts appear as a characteristic sign of the condition in these women, especially those with phenotype D.

Ovarian cysts are usually of the follicular or luteal type. The first are more common and form following an incorrect release on the oocyte from the follicle, remaining trapped inside and subsequently fills with liquid to form a follicular cyst, which often disappears within a few menstrual cycles. Luteal cysts, instead form when the follicle closes following the expulsion of the oocyte, retaining various kinds of fluid and blood inside. These are more dangerous and less common than follicular cysts.

Women with PCOS phenotype D who present a polycystic ovary upon ultrasound examination, unlike the multimolecular ovary, demonstrate over 12 total cysts, with dimensions between 2 and 9 mm. In detail, the cysts present a characteristic crown-like arrangement across the ovary and are associated with an increase in ovarian volume.

Currently, the most commonly recommended remedy for the treatment of ovarian cysts, as described above, are oral contraceptives, that are prescribed in order to induce reabsorption and/or a reduction of the volume of said cysts.

Therefore, a therapy that allows for the prevention of these conditions and the regression of ovarian cysts in women with PCOS phenotype D, adds a further novel element to the present finding. In this complex pathological context such as is the case of PCOS, the authors have found an approach that utilized three natural functional components and resolves one or the more important issues for phenotype D.

The inventors of the present invention have identified an unanticipated discovery that the composition, according to the invention, that consists of diosgenin, or in the form on an extract, preferably in the form of an abstract of Dioscorea villosa, vitamin D, and α-lactalbumin, displays a synergistic effect. With the expression “synergistic effect” it is intended that the given effect of the composition according to the invention is superior in respect to the sum of effects displayed by the single components when administered separately.

The three components of the present invention work in a synergistic manner in treating the condition of anovulation, and formation of ovarian cysts that provoke alterations in the menstrual cycle and reduce female fertility, in particular favoring the normalization of the duration of the follicular phase and the physiology of the ovulatory process. The effect derived from the combination of these three components is superior to those effects that the single components display and the effect is not simply an additive effect given from the combination of three components in a singular formulation. Furthermore, the improvement probably of conception is not a result that can be obtained through only the use of the single components described in the present invention but rather represents a completely innovative activity associated with the combination of these components.

The present invention represents an important discovery in the field of remedies commonly adapted for the treatment of PCOS, because it constitutes a specific approach designed for a subclass of these patients who require a more targeted therapy than those which are currently available.

Furthermore, the invention describes a completely natural remedy and provides a valid alternative to non-specific hormonal treatments for PCOS phenotypes.

### Definitions

The term diosgenin as used here indicates the natural product, spirost-5-en-3-ol, (3B, 25R)-(PCI, ACE; CAS Registery number: 512-04-9), a sapogenin present mainly in the tubers of Dioscorea and corresponding to the compound of formula (I):

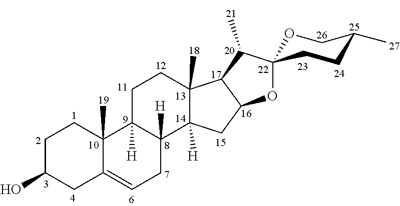

Diosegin is primarily contained in plants belong to the Dioscoreaceae family, for example Dioscorea villosa, Dioscorea alata, Dioscorea bulbifera, Dioscorea composita, Dioscorea oppostifolia, Dioscorea polystachy, Dioscorea hirticaulis, Dioscorea nipponica, Dioscorea zingibergensis, Dioscorea deltoidea, Dioscorea collettii, Dioscorea semptemloba, and is commonly extracted from plant parts, in particularly the rhizome or roots. Synonyms of Dioscorea include Atlantic yam, common wild yam, wild yam, yellow yam, colic root, and the rheumatism root. Other plants from which it is possible to extract diosgenin include for example the species Trigonella, Costus, and Smilax.

Derivatives of diosgenin include derivatives at the C3 position, in particular compounds derived from the functionalization of the hydroxy group, in particular its esters, ethers, or its glucopyranosides such as dioscin ((β-D-Glucopyranoside, (33,25R)-spirost-5-en-3-yl O-6-deoxy-α-L-mannopyranosyl-(1→2)-[α-L-mannopyranosyl-(1→4)]-(9CI, ACI); CAS No. 19057-60-4). Other derivatives include molecules derived from opening of the spirocycle further functionalized at the C26 position. For a review on the derivatives, see for example Food Sci. Technol, Campinas, v42, e94521, 2022 (DOI: https://doi.org/10.1590/fst.94521).

The expression vitamin D, as it is used here, includes vitamin D2, vitamin D3, ergocalciferol, cholecalciferol, and calciferol.

α-lactalbumin is a milk serum protein and, as it is used here, the expression α-lactalbumin includes the isolated protein, whey protein isolates and hydrosylates of α-lactalbumin itself.

In the present context, the term “approximately”, when referring to a particular value, for example an endpoint or endpoints of a range, includes and describes, in addition to the specified value itself, some variation around the specified value. Such variations may, for example, derive from normal variations in measurement, for example in the weighting or aliquoting of various substances by methods known to a skilled person. The term “approximately” shall be understood as inclusive and describe a range of variability above and below a specific stated value, said percentage values being relative to the specified stated value, as follows. The term “approximately” can include a variability between a minimum of +0.1% and a maximum of +5.0%. The term “approximately” when referring to a particular cited value, may include and describe that particular exact value, regardless of any explicit mention of this particular exact value being included; even in the absence of an explicit indication, the term “approximately” includes the particular exact specified value, this particular exact value is still included in the range of variation created by the term “approximately”, and is therefore described.

As described above, the present invention refers to diosgenin in combination and/or association with at least one of the following agents: vitamin D and α-lactalbumin for use in the regularization of the menstrual cycle and the reduction of ovarian cysts, preferably for the regularization of the menstrual cycle and the reduction of ovarian cysts in women affected by polycystic ovarian syndrome, preferably in PCOS phenotype D. In a correlated aspect, the present invention regards a method of control and/or regularization of the menstrual cycle and the reduction of ovarian cysts, preferably for the regularization of the menstrual cycle and the reduction of ovarian cysts in women affected by polycystic ovarian syndrome, preferably in PCOS phenotype D.

In another correlated aspect, the present invention regards the use of diosgenin, vitamin D, and α-lactalbumin in the production of a product for the regularization of the menstrual cycle and the reduction of ovarian cysts, preferably for the regularization of the menstrual cycle and the reduction of ovarian cysts in women affected by polycystic ovarian syndrome, preferably in PCOS phenotype D. The agents in the present invention may exist and be used according to the invention in any form of salt, solvent, stereoisomer, zwitterion and/or isotope.

The salts of the agents in the present invention are, preferably, pharmaceutically acceptable. Suitable pharmaceutically acceptable salts include conventional non-toxic salts obtained by salification of an agent of the present invention with inorganic acids (for example hydrochloric, hydrobromic, sulfuric or phosphoric acids) or with organic acids (for example acetic, propionic, succinic, benzoic, sulphanilic, 2-acetoxy-benzoic, cinnamic, mandelic, salicylic, glycolic, lactic, oxalic, malic acids, maleic, malonic, fumaric, tartaric, citric, p-toluenesulfonic, methanesulfonic, ethanesulfonic or naphthalenesulfonic). For a review on suitable pharmaceutically acceptable see for example Berge S. M. et al., J. Pharm. Sci. 1977, 66, 1-19.

Furthermore, pharmaceutically accepted salts can be formed with a suitable inorganic or organic base such as triethylamine, ethanolamine, triethanolamine, dicyclohexylamine, ammonium hydroxide, pyridine, glucosamine, galactosamine. The term “inorganic base” as used herein, has its ordinary meaning as understood by a person of ordinary skill in the art and generally refers to an inorganic compound that can serve as a proton acceptor. Also, the term “organic base” as used herein, has its ordinary meaning as understood by a person of ordinary skill in the art and generally refers to an organic compound that can serve as a proton acceptor. Other suitable pharmaceutically acceptable salts include pharmaceutically acceptable salts of alkali metals or alkaline earth metals such as sodium, potassium, calcium, or magnesium salts. The invention regards the use of all possible stochiometric and non-stochiometric forms of salts of the agents of the present invention.

Furthermore, the agents in the present invention may exist in non-solvated forms and solvated forms with pharmaceutically acceptable solvents such as water ethanol and the like.

In the present context, the term “combination” refers to the pairing of diosgenin with at least one of the following agents: vitamin D and α-lactalbumin and for them to jointly administered to a subject as part of a given treatment regimen. A given treatment regimen may consist of multiple and repeated joint administration of diosgenin, vitamin D, and α-lactalbumin for a predetermined period, for example for 1 month, 2 months, 3 months, 4 months, 5 months, 6 months, 7 months, 8 months, 9 months, 10 months, 11 months 12 months or more, a considered period of time ranging from 6 to 12 months would be preferred. The joint administration or combination of diosgenin, vitamin and α-lactalbumin may be repeated multiple times a day, for example once, twice, three times, four times or five or more times, a frequency of approximately once/twice a day would be preferred.

In the present context, the term “composition” includes and describes any physical entity comprising or consisting of, or essentially consisting of, the respective described substances, for example comprising or consisting, or essentially consisting of, diosgenin and at least one of the following agents: vitamin D and α-lactalbumin. The physical form of the composition is not limited. For example, the term “composition” includes and describes a powder in which each of the described substances is in powdered form. As another example, the term “composition” also includes and describes a liquid solution in which the described substances are present in a solubilized form. As another example the term “composition” also includes and describes an emulsion in which the described substances are present. As another example the term “composition” also includes and describes a suspension in which the described substances are present. As another example the term “composition” also includes and describes mixtures in which diosgenin is in a form, for example a solid such as a powder, while at least one of the following agents: vitamin D and α-lactalbumin is in another form, for example a liquid. In particular, the term “pharmaceutical composition” as it is defined below, may be formulated for any desired administration. As it is used and described here, the term “composition” may furthermore be a composition adapted for oral release, for example in the form of a tablet including, but not limited to, an effervescent tablet or a multilayer tablet, a powder, for example in the form of a sachet, a hard capsule, a soft gel capsule, a syrup, a drinking liquid, a lozenge for example a gummy tablet, a balm or another liquid preparation, an ointment, a cream, a gel. In some realized forms particularly preferred by the invention, the “composition” includes diosgenin and at least one of the following agents: vitamin D and α-lactalbumin may be in the form of a soft gel. In certain other realized forms particularly preferred by the invention, the “composition” includes diosgenin and at least one of the following agents: vitamin D and α-lactalbumin in the form of a powder. In certain other realized forms particularly preferred by the invention, the “composition” includes diosgenin and at least one of the following agents: vitamin D and α-lactalbumin in the form of a cream, gel or ointment. The term “composition” can also be a composition adapted for another non oral release, for example in the form of a suppository, a tablet, a hard capsule, a soft gel capsule, a cream, a gel, a patch, a liquid. Further dosage forms of the composition, as well as the indicated routes of administration are given below. The compositions of the invention, including pharmaceutical compositions, may also include at least one pharmaceutically acceptable ingredient. While the inventive composition, including the pharmaceutical composition, may itself be administered to a subject, it will be understood that the addition of one or more pharmaceutically acceptable ingredients, in addition to diosgenin: vitamin D and α-lactalbumin may be advantageous in making the composition more adapted for direct administration in a subject by a predetermined route. Consequently, the “pharmaceutical composition”, may be formulated for diosgenin and at least one of the following agents: vitamin D and α-lactalbumin, a pharmaceutically acceptable ingredient that makes the composition more adapted or particularly adapted for direct administration in a subject via a predetermined route, without further tests. This adequacy may cover a number of different routes of administration including oral, parenteral, transmucosal, vaginal or perivaginal, topical, transdermal or intravesical.

### EXAMPLES

The present invention includes the combination and/or composition of diosgenin, or in the form of a plant extract, preferably said plant belonging to the Discoreaceae, preferably in the form of an extract of Dioscorea villosa, with at least one of the following agents: vitamin D, α-lactalbumin, capable of exerting a more advantageous effect in respect of the use of the singular substances in the regularization of the menstrual cycle in the reduction of ovarian cysts in women effected by PCOS.

This represents a new and unexpected approach to the prevention/treatment of PCOS phenotype D.

### Example 1

An exemplified composition of the present invention is in the form of oblong tablet (10 mm in diameter), of approximately 550 mg by weight, with an expected storage period of 36 months (if stored in a fresh, dry, and dark place) and having the following composition:

TABLE 1
Composition of example 1

| Ingredients | mg/tablet | Intake per tablet |
|---|---|---|
| DIOSCOREA rhizome | 300 | 60 mg of Diosgenin |
| Dry extract 20% diosgenin | | |
| Vitamina D3 100.000 IU/g | 10 | 25 mcg |
| α-lactalbumin 65% | 77 | 50 mg of α-lactalbumin |
| Microcrystalline cellulose | 138.00 | |
| Silicon dioxide | 15.00 | |
| Magnesium salts of fatty acids | 10.00 | |
| TOTAL | 550 mg | |

The amount of the ingredients listed in the column “mg/tablet” of Table 1 refers to the amount of ingredient used to prepare a tablet. The amount of ingredient list in the column “intake per tablet” in Table 1 refers to the amount of active ingredient present in a tablet.

It is recommended to take two tablets per day.

### Example 2

A further exemplified composition of the present invention is in the form of oblong tablet (10 mm in diameter), of approximately 550 mg by weight, with an expected storage period of 36 months (if stored in a fresh, dry, and dark place) and having the following composition:

TABLE 2
Composition of example 2

| Ingredients | mg/tablet | Intake per tablet |
|---|---|---|
| DIOSCOREA rhizome | 300 | 18 mg of Diosgenin |
| Dry extract 6% diosgenin | | |
| Vitamina D3 100.000 IU/g | 10 | 25 mcg |
| α-lactalbumin 65% | 77 | 50 mg of α-lactalbumin |
| Microcrystalline cellulose | 138.00 | |
| Silicon dioxide | 15.00 | |
| Magnesium salts of fatty acids | 10.00 | |
| TOTAL | 550 mg | |

The amount of the ingredients listed in the column “mg/tablet” of Table 2 refers to the amount of ingredient used to prepare a tablet. The amount of ingredient list in the column “intake per tablet” in Table 1 refers to the amount of active ingredient present in a tablet.

It is recommended to take two tablets per day.

### Example 3

Another exemplified composition of the present invention is in the form of a sachet containing a soluble formulation (approximately 2200 mg by weight) and having the following composition:

TABLE 3
Composition of example 3

| Ingredients | mg/sachet | Intake per sachet |
|---|---|---|
| DIOSCOREA rhizome | 300 | 18 mg of Diosgenin |
| Dry extract 6% diosgenin | | |
| Vitamina D3 100.000 IU/g | 11 | 25 mcg + 10% |
| α-lactalbumin 65% | 77 | 50 mg of α-lactalbumin |
| Maltodextrin | 1562 | |
| Silicon dioxide | 30 | |
| Flavoring | 100 | |
| Sucralose | 20 | |
| Citric acid | 100 | |
| TOTAL | 2200 mg | |

The amount of the ingredients listed in the column “mg/sachet” of Table 3 refers to the amount of ingredient used to prepare a sachet. The amount of ingredient list in the column “intake per sachet” in Table 3 refers to the amount of active ingredient present in a sachet.

It is recommended to take two sachets per day.

### Example 4. Testing the Effect of the Administration of Dioscorea villosa, Vitamin D, and α-Lactalbumin on the Expression of IGF-1 in Bovine Theca and Granulosa Cells

The anti-proliferative activity of Dioscorea villosa, Vitamin D, and α-lactalbumin was investigated (alone or in combination) in cultured bovine theca and granulosa cells (co-culture). To investigate the properties of these natural molecules, the inventors evaluated their effect on cell viability, proliferation, and apoptosis.

### Phase I

Bovine theca and granulosa were co-cultured, (DMEM, High glucose 5% FBS, 4% Gentamicin, 1% Pen/Strep, 1% Amphotericin B, 1% Glutamine) for 7 days. The cells were grown in petri dishes and treated shortly before reaching 80% confluence.

The experimental conditions used for the treatments are as follows:
1_control (only DMEM complete)
2_stimulation with FSH (46 h), followed by LH 1.5 UI/mL, and insulin 100 ng/ml for 10 minutes.
3_stimulation with FSH (46 h), followed by LH 1.5 UI/ml, and insulin 100 ng/ml for 10 minutes. Administration of IGF_1 (100 ng/ml, for 48 h).

The expression of insuline like growth factor IGF-1 and IGF-BP1 insulin like growth factor binding protein 1 were both measured by Real Time PCR and western blot, to investigate the effects of the treatment with the raw materials; namely, Dioscorea villosa, Vitamin D and α-lactalbumin alone or in combination.

The results were measured as 2^{-ΔΔCt} for the mRNA assay. Both for the Real Time PCR and western blot analyses, the data are expressed as fold increases in relation to the housekeeping genes. Proliferation (ki67 western blot assay) and apoptosis (caspase-9 western blot assay) assessed the expression as per the experimental conditions reported above.

### Phase 2

Co-cultured bovine theca and granulosa cells; grown as described in phase 1. The expression of IGF-1 and IGF-BP1 were analyzed after treatment with Dioscorea villosa. The experimental conditions used for treatments were as follows:
1_“phase 2 control”: stimulation with FSH (46 h), followed by LH 1.5 UI/ml, and insulin 100 ng/ml for 10 minutes. Administration of IGF_1 (100 ng/ml, for 48 h).
2_phase 2 control+Dioscorea villosa 75 μM for 24 h.
3_phase 2 control+Dioscorea villosa 100 μM for 24 h

The expression of IGF-1 and IGF-BP1 were both measured by Real Time PCR and western blot, to investigate the effects of the treatment with the raw materials; namely, Dioscorea villosa, Vitamin D and α-lactalbumin alone or in combination.

The results were measured as 2-4Act for the mRNA assay. Both for the Real Time PCR and western blot analyses, the data are expressed as fold increases in relation to the housekeeping genes. Proliferation (ki67 western blot assay) and apoptosis (caspase-9 western blot assay) assessed the expression as per the experimental conditions reported above.

### Phase 3

Co-cultured bovine theca and granulosa cells; grown as described in phase 1. The expression of IGF-1 and IGF-BP1 were analyzed after treatment with Dioscorea villosa, Vitamin D and α-lactalbumin alone or in combination.

The experimental conditions used for treatments were as follows:
1_“phase 3 control”: stimulation with FSH (46 h), followed by LH 1.5 UI/ml, and insulin 100 ng/ml for 10 minutes. Administration of IGF_1 (100 ng/ml, for 48 h);
2_phase 3 control+Dioscorea villosa 100 μM for 24 h;
3_phase 3 control+Vitamin D 20 μM for 24 h;
4_phase 3 control+α-lactalbumin 10 nM for 24 h;
5_phase 3 control+Dioscorea villosa 100 μM+Vitamin D 20 μM for 24 h;
6_phase 3 control+Dioscorea villosa 100 μM+α-lactalbumin 10 nM for 24 h;
7_phase 3 control+Vitamin D 20 μM+α-lactalbumin 10 nM for 24 h;
8_phase 3 control+Dioscorea villosa 100 μM+Vitamin D 20 μM+α-lactalbumin 10 nM for 24 h.

The expression of IGF-1 and IGF-BP1 were both measured by Real Time PCR and western blot, to investigate the effects of the treatment with the raw materials; namely, Dioscorea villosa, Vitamin D and α-lactalbumin alone or in combination.

The results were measured as 2-44Ct for the mRNA assay. Both for the Real Time PCR and western blot analyses, the data are expressed as fold increases in relation to the housekeeping genes. Proliferation (ki67 western blot assay) and apoptosis (caspase-9 western blot assay) assessed the expression as per the experimental conditions reported above.

### Results

Co-cultured bovine theca and granulosa cells demonstrated a strong proliferative effect following treatment with IGF-1 (48 h): suggesting an overlap with the data reported by Dai and colleagues in mouse 3D follicle models. The described effect was also associated with a reduction in the rate of apoptosis observed in the cell culture.

Treatment with Dioscorea villosa significantly inhibited IGF-1 expression, favoring the increase in IGF-BP-1 protein levels. The combination of the three raw materials used in this experimental setting induced a reduction of IGF-1 and a simultaneous increase in the IGF-BP-1, which was higher than the effect produced observed when the raw materials were used alone. A similar result was observed when comparing treatment with the combination of all three raw materials with respect to the various combinations consisting of two out of three raw materials (points 5, 6 and 7 of phase 3).

### BIBLIOGRAPHY

1. Rosenfield, R. L. and D. A. Ehrmann, The Pathogenesis of Polycystic Ovary Syndrome (PCOS): The Hypothesis of PCOS as Functional Ovarian Hyperandrogenism Revisited. Endocr Rev, 2016. 37 (5): p. 467-520.
2. Revised 2003 consensus on diagnostic criteria and long-term health risks related to polycystic ovary syndrome. Fertil Steril, 2004. 81 (1): p. 19-25.
3. Papadakis, G., et al., Tailoring treatment for PCOS phenotypes. Expert Rev Endocrinol Metab, 2021. 16 (1): p. 9-18.
4. Mumusoglu, S. and B. O. Yildiz, Polycystic ovary syndrome phenotypes and prevalence: Differential impact of diagnostic criteria and clinical versus unselected population. Current Opinion in Endocrine and Metabolic Research, 2020. 12: p. 66-71.
5. Li, F., et al., Oral Contraceptive Use and Increased Risk of Stroke: A Dose-Response Meta-Analysis of Observational Studies. Frontiers in Neurology, 2019. 10.

6 Facchinetti, F., et al., Short-term effects of metformin and myo-inositol in women with polycystic ovarian syndrome (PCOS): a meta-analysis of randomized clinical trials. Gynecol Endocrinol, 2019. 35 (3): p. 198-206.
7. Tang, Z. R., et al., Estrogen-Receptor Expression and Function in Female Reproductive Disease. Cells, 2019. 8 (10).
8. Dai, S., et al., Effects of IGF-1 on the Three-Dimensional Culture of Ovarian Preantral Follicles and Superovulation Rates in Mice. Biology (Basel), 2022. 11 (6).
9. Palomba, S., T. T. Piltonen, and L. C. Giudice, Endometrial function in women with polycystic ovary syndrome: a comprehensive review. Hum Reprod Update, 2021. 27 (3): p. 584-618.
10. Monastra, G., et al., Vitamin D: a steroid hormone with progesterone-like activity. Eur Rev Med Pharmacol Sci, 2018. 22 (8): p. 2502-2512.
11. Morgante, G., et al., PCOS Physiopathology and Vitamin D Deficiency: Biological Insights and Perspectives for Treatment. J Clin Med, 2022. 11 (15).
12. Graham, M. E., et al., Gut and vaginal microbiomes on steroids: implications for women's health. Trends Endocrinol Metab, 2021. 32 (8): p. 554-565.

1. A composition comprising diosgenin or a salt or a derivative thereof and a further agent selected from vitamin D and α-lactalbumin.

2. The composition according to claim 1 wherein diosgenin is comprised in powdered parts of plants or in an extract of said parts of plants, wherein said powdered parts of plants or extract thereof are obtained from plants preferably belonging to the Dioscoreaceae family, preferably Dioscorea villosa, Dioscorea alata, Dioscorea bulbifera, Dioscorea composita, Dioscorea oppostifolia, Dioscorea polystachy, Dioscorea hirticaulis, Dioscorea nipponica, Dioscorea zingibergensis, Dioscorea deltoidea, Dioscorea collettii, Dioscorea semptemloba, more preferably said powdered parts of plants or said extract comprise diosgenin in an amount from about 1% by weight to about 50% by weight, preferably from about 1% to about 30% by weight, preferably about 20% by weight or about 15% by weight or about 10% by weight or about 6% by weight.

3. The composition according to claim 2 comprising:
a) Parts of plants, preferably powdered parts of plants or an extract thereof obtained from Dioscorea villosa in an amount comprised from 1 to 600 mg, preferably from 200 mg to 600 mg, preferably from 200 mg to 400 mg, preferably from 250 mg and 350 mg, more preferably in the amount of 300 mg;
b) Vitamin D in an amount comprised between 1 and 50 μg, i.e. from 40 to 2000 UI, preferably in an amount comprised between 10 and 40 μg, i.e. from 400 to 1600 UI, preferably in the amount comprised between 20 and 30 μg, i.e. from 800 and 1200 UI, more preferably in the amount of 25 μg i.e. 1000 UI; and/or
c) α-lactalbumin in an amount comprised between 1 and 100 mg, preferably in an amount between 25 and 75 mg, preferably comprising α-lactalbumin in the amount between 40 and 60 mg, preferably comprising α-lactalbumin in the amount of 50 mg.

4. The composition according to claim 1 comprising diosgenin in an amount between 0.2 mg and 120 mg, preferably from 40 mg to 120 mg, preferably from 40 mg to 80 mg, preferably from 50 mg to 70 mg, preferably in the amount of about 60 mg.

5. The composition according to claim 1 comprising diosgenin in an amount between 0.06 mg and 36 mg, preferably from 12 mg to 24 mg, preferably from 15 mg to 21 mg, preferably in the amount of about 18 mg.

6. The composition according to claim 1 wherein vitamin D is selected from ergocalciferol, cholecalciferol, calciferol, calcitriol, calcifediol, and/or wherein α-lactalbumin is selected from α-lactalbumin, lactalbumin hydrolysates, whey protein isolate.

7. The composition according to claim 1 further comprising at least an excipient or diluent, preferably for oral administration and/or topical administration, preferably in the form of a tablet, a hard capsule, a soft gel capsule, a powder, a syrup, a cachet, a pill, a bar, a gel, a cream, an ointment, preferably said excipient and/or diluent is selected from group comprising of: calcium phosphate, dicalcium phosphate, microcrystalline cellulose, silicon dioxide, sucrose, gum arabic, corn starch, medium chain triglycerides, tricalcium phosphate, cross-linked sodium carboxymethylcellulose, hydroxypropyl methylcellulose, polyethylene glycol, titanium dioxide, polyvinylpyrrolidone, talc, erythritol, xylitol, steviol glycosides and sucralose, magnesium salt of fatty acids, preferably said magnesium salt of fatty acids is magnesium stearate.

8. A method for the regularization of the menstrual cycle and in the reduction of ovarian cysts, comprising administering an effective amount of the composition of claim 1 to a woman in need thereof.

9. A dermo-cosmetic product or medical device or supplement or dietary product, or formulation comprising the composition according to claim 1.

10. (canceled)

11. A combination comprising diosgenin or its salt or derivative thereof and at least one further agent selected from vitamin D and α-lactalbumin.

12. The combination according to claim 11 wherein diosgenin is comprised of powdered plant parts or in an extract of said plant parts, in which said powdered plants parts or plant extracts are obtained from plants belonging to the Dioscoreaceae family, preferably Dioscorea villosa, Dioscorea alata, Dioscorea bulbifera, Dioscorea composita, Dioscorea oppostifolia, Dioscorea polystachy, Dioscorea hirticaulis, Dioscorea nipponica, Dioscorea zingibergensis, Dioscorea deltoidea, Dioscorea collettii, Dioscorea semptemloba, more preferably said plant parts or plant extract comprise diosgenin in an amount between approximately 1% by weight to approximately 50% by weight, preferably between 1% and 30% by weight, about 20% by weight, about 15% by weight, about 10% by weight or about 6% by weight.

13. The combination according to claim 12 comprising:
a) Plant parts, preferably powdered parts or extract obtained from Dioscorea villosa in an amount including between 1 and 1200 mg/day, preferably between 200 mg and 1000 mg/day, preferably between 200 mg and 800 mg/day, preferably between 400 mg and 800 mg/day or between 300 and 700 mg/day, preferably in the amount of 600 mg/day, preferably in the amount of 300 mg/day;
b) Vitamin D in an amount including between 1 and 100 μg/day (from 40 to 4000 UI/day), preferably in the amount including between 20 and 80 μg/day (from 800 to 3200 UI/day), preferably in the amount including between 25 and 60 μg/day (from 1000 to 2400 UI/day), preferably in the amount of 25 μg (1000 UI) per day, more preferably in the amount of 50 μg (2000 UI) per day; and/or
c) α-lactalbumin in an amount including between 1 and 200 mg/day, preferably in an amount between 25 and 150 mg/day, preferably in an amount between 50 and 120 mg/day, preferably in the amount of 50 mg/day, more preferably in the amount of 100 mg/day.

14. The combination according to claim 11 comprising diosgenin in an amount between 0.2 mg and 240 mg/day, preferably between 40 mg and 200 mg/day, preferably between 80 mg and 200 mg/day, preferably between 80 mg and 160 mg/day, preferably about 120 mg/day.

15. The combination according to claim 11 comprising diosgenin in amount between 0.06 mg and 72 mg/day, preferably between 12 mg and 48 mg/day, preferably between 15 mg and 42 mg/day, preferably about 18 mg/day, more preferably about 36 mg/day.

16. The combination according to claim 11 wherein vitamin D is selected from: ergocalciferol, cholecalciferol, calciferol, calcitriol, calcifediol, and/or α-lactalbumin is selected from α-lactalbumin, lactalbumin hydrolysates, whey protein isolate.

17. The combination according to claim 11 wherein diosgenin and the further agent or agents are administered simultaneous and/or orally and/or via topical application, once and/or twice a day.

18. The method of claim 8, wherein the woman is affected by polycystic ovarian syndrome (PCOS).

19. The method of claim 18, wherein the PCOS is of Phenotype-D.
